(19) United States
(12) Patent Application Publication
(10) Pub. No.: US 2023/0202906 A1
(43) Pub. Date: Jun. 29, 2023

(54) LITHIUM SILICATE GLASS CERAMIC COMPRISING TIN

(71) Applicant: Ivoclar Vivadent AG, Schaan (LI)

(72) Inventors: Marc DITTMER, Feldkirch (AT); Christian RITZBERGER, Grabs (CH); Markus RAMPF, Seewis-Dorf (CH); Katrin SULSER, Trübbach (CH)

(21) Appl. No.: 18/068,308

(22) Filed: Dec. 19, 2022

(30) Foreign Application Priority Data
Dec. 23, 2021 (EP) 21217422.1

(51) Int. Cl. C03C 3/083; C03C 3/097; C03C 4/00

(52) U.S. Cl. CPC C03C 3/083; C03C 3/097; C03C 4/0021; C03C 4/0028

(57) ABSTRACT

Lithium silicate glass ceramics and precursors thereof are described, which contain tin and are characterized by very good mechanical and optical properties and can be used in particular as restorative materials in dentistry.

### CROSS-REFERENCE TO RELATED APPLICATIONS

This application claims priority to European Patent Application No. 21217422.1 filed on Dec. 23, 2021, the disclosure of which is incorporated herein by reference in its entirety.

### TECHNICAL FIELD

The invention relates to lithium silicate glass ceramic comprising tin, which is particularly suitable for use in dentistry and preferably for producing dental restorations, and to precursors for producing this glass ceramic.

### BACKGROUND

Glass ceramics containing tin are known from the prior art.

EP 1 985 591 and corresponding U.S. Pat. No. 8,283,268 B2, which US patent is hereby incorporated by reference in its entirety, describe glass ceramics which can be colored by metal colloids. Possible metal colloid formers are compounds of the metals Au, Ag, As, Bi, Nb, Cu, Fe, Pd, Pt, Sb and Sn. The glass ceramics are, in particular, lithium aluminosilicate glass ceramics or magnesium aluminosilicate glass ceramics containing high amounts of aluminum oxide of at least 18.0 wt.-% and significant amounts of antimony oxide and arsenic oxide, which are harmful to health.

WO 03/050053 and corresponding US 2005142077, which US published application is hereby incorporated by reference in its entirety, and WO 03/050051 and corresponding U.S. Pat. No. 7,141,520, which US patent is hereby incorporated by reference in its entirety, describe antimicrobial glass ceramic powders that can be used in the field of dental care, for example as a component of mouthwash, toothpaste or dental floss. To enhance the antimicrobial properties, antimicrobially active ions such as Ag, Au, I, Ce, Cu, Zn and Sn may be present. The glass ceramics have alkali earth alkali silicates and/or alkaline earth silicates, in particular NaCa silicates and Ca silicates, as the main crystalline phase.

WO 2005/058768 and corresponding U.S. Pat. No. 7,157,149, which US patent is hereby incorporated by reference in its entirety, disclose bodies of lithium aluminosilicate glass ceramics, which are particularly suitable for the manufacture of cooking hobs. The bodies have a surface layer with a higher content of crystallization-promoting chemical elements from the group of Zn, Cu, Zr, La, Nb, Y, Ti, Ge, V and Sn. As the main crystalline phase, the glass ceramics contain a high quartz solid solution phase.

EP 1 688 397 describes lithium silicate glass ceramics containing small amounts of zinc oxide as well as high amounts of 2.0 to 5.0 wt.-% nucleating agent. The nucleating agent for forming lithium metasilicate is in particular selected from P₂O₅ and compounds of the elements Pt, Ag, Cu and W and it is preferably P₂O₅. Accordingly, P₂O₅ is also used as the nucleating agent in all the specifically disclosed glass ceramics, which, in addition to lithium silicate, also leads to the formation of lithium phosphate as crystal phase. However, lithium phosphate crystals can impair the mechanical and/or optical properties of lithium silicate glass ceramics.

WO 2013/053866 and corresponding U.S. Pat. No. 9,695,082, which US patent is hereby incorporated by reference in its entirety, describe lithium silicate glass ceramics containing tetravalent metal oxides, such as tin oxide. Metals and in particular Ag, Au, Pt and Pd and particularly preferably P₂O₅ are used as nucleating agents for the formation of lithium silicate. However, the use of P₂O₅ as nucleating agent results in the formation of undesirable lithium phosphate as crystal phase. Furthermore, the glass ceramics contain only very small amounts of the monovalent metal oxides K₂O and Na₂O and are preferably essentially free of these metal oxides.

EP 3 696 149 A1 and corresponding U.S. Ser. No. 11/440,833, which US patent is hereby incorporated by reference in its entirety, describe fluorescent glass ceramics and glasses which contain cerium and tin to produce fluorescence and P₂O₅ as nucleating agent. In this context, the tin serves for the desired adjustment of the equilibrium between Ce³⁺ and Ce⁴⁺ ions, whereby the desired fluorescence and the desired coloration of the glass ceramic are achieved. The use of P₂O₅ as a nucleating agent can in turn result in the presence of undesirable phosphate crystal phases in the glass ceramics.

In summary, the known glass ceramics do not possess the properties desirable for a dental restorative material or they contain high amounts of P₂O₅, which can lead to the formation of undesirable crystal phases, such as phosphate phases or cristobalite, which in turn can impair in particular the mechanical and/or optical properties desired for a restorative material.

### SUMMARY

The invention is therefore based on the problem of providing a glass ceramic with a combination of very good mechanical and optical properties. The glass ceramic should also be easy to process into dental restorations and thus be excellently suited as a restorative dental material.

This problem is solved by the lithium silicate glass ceramic according to the claims. Also subject to the invention are the starting glass according to the claims, the processes according to the claims, and the use.

### BRIEF DESCRIPTION OF THE DRAWINGS

Further details, advantages and features will be apparent from the following description with reference to the drawing, in which:

FIG. 1 shows four platelets of a glass ceramic.

### DETAILED DESCRIPTION

The lithium silicate glass ceramic according to the invention is characterized by the fact that it comprises 0.01 to 4.5, preferably 0.03 to 3.0, particularly preferably 0.1 to 2.0 and most preferably 0.2 to 1.5 wt.-% tin, calculated as SnO₂.

Surprisingly, the glass ceramic according to the invention shows an advantageous combination of mechanical and optical properties desirable for a restorative dental material. The glass ceramic has a high strength and fracture toughness, and it can be easily given the shape of a dental restoration by in particular machining.

It is surprising that the use of P₂O₅ as the usual nucleating agent for lithium silicate glass ceramics is not necessary to achieve these properties. It is assumed that in the glass ceramic according to the invention, the tin present serves as the nucleating agent. It is particularly surprising that even small amounts of tin are effective.

The glass ceramic according to the invention can also have very high amounts of lithium silicate crystal phases of, for example, more than 65 wt.-%, and it is again assumed that the tin present as nucleating agent is essentially responsible for this. Such high contents of lithium silicate crystal phases are usually not producible when P₂O₅ is used as nucleating agent.

The glass ceramic according to the invention also preferably has only small amounts of further crystal phases, e.g. lithium phosphate or cristobalite. The formation of large amounts of such further crystal phases frequently occurs with the use of large amounts of P₂O₅ as nucleating agent, which has been common up to now, and these further crystal phases can have a negative effect on the mechanical and/or optical properties of lithium silicate glass ceramics. In addition, lithium is consumed by the formation of lithium phosphate crystals and is thus no longer available for the formation of lithium silicate. It is the lithium silicate that plays an essential role, especially for the excellent mechanical properties of lithium silicate glass ceramics. Accordingly, the glass ceramic according to the invention is also advantageous in this respect.

The glass ceramic according to the invention comprises in particular 65.0 to 89.0, preferably 68.0 to 83.0, particularly preferably 75.0 to 81.0 and most preferably 77.0 to 80.0 wt.-% SiO₂.

It is further preferred that the glass ceramic according to the invention comprises 10.0 to 21.0, preferably 11.0 to 20.0, more preferably 13.0 to 19.0, and most preferably 14.0 to 18.0 wt.-% Li₂O. It is assumed that Li₂O also lowers the viscosity of the glass matrix and thus promotes crystallization of the desired crystal phases.

It is also preferred that the glass ceramic comprises 0 to 7.0 and preferably 1.0 to 6.0 wt.-% oxide of monovalent elements Me^{I}₂O selected from the group of K₂O, Na₂O, Rb₂O, Cs₂O and mixtures thereof.

Particularly preferably, the glass ceramic comprises at least one and, in particular, all of the following oxides of monovalent elements Me^{I}₂O in the amounts indicated:

| Component | Wt.-% |
|---|---|
| K₂O | 0 to 6.0 |
| Na₂O | 0 to 6.0 |
| Rb₂O | 0 to 5.0 |
| Cs₂O | 0 to 4.0 |

In a particularly preferred embodiment, the glass ceramic according to the invention comprises 1.0 to 5.0, preferably 1.2 to 4.5, more preferably 1.5 to 4.0, and most preferably 1.5 to 2.5 wt.-% K₂O.

Furthermore, it is preferred that the glass ceramic comprises 0 to 15.0, preferably 0 to 10.0, and most preferably 0 to 8.0 wt.-% oxide of divalent elements Me^{II}O selected from the group of CaO, MgO, SrO, ZnO, and mixtures thereof.

In another preferred embodiment, the glass ceramic comprises less than 2.0 wt.-% of BaO. In particular, the glass ceramic is substantially free of BaO.

Preferably, the glass ceramic comprises at least one, and in particular all, of the following oxides of divalent elements Me^{II}O in the amounts indicated:

| Component | Wt.-% |
|---|---|
| CaO | 0 to 10.0, in particular 0 to 8.0 |
| MgO | 0 to 8.0, in particular 0 to 6.0 |
| SrO | 0 to 15.0, in particular 0 to 12.0 |
| ZnO | 0 to 12.0, in particular 0 to 10.0 |

Further preferred is a glass ceramic comprising 0 to 12.0, preferably 0.1 to 10.0, and most preferably 1.0 to 8.0 wt.-% oxide of trivalent elements Me^{III}₂O₃ selected from the group of Al₂O₃, B₂O₃, Y₂O₃, La₂O₃ and mixtures thereof.

Particularly preferably, the glass ceramic comprises at least one, and in particular all, of the following oxides of trivalent elements Me^{III}₂O₃ in the amounts indicated:

| Component | Wt.-% |
|---|---|
| Al₂O₃ | 0 to 6.0 |
| B₂O₃ | 0 to 5.0 |
| Y₂O₃ | 0 to 8.5 |
| La₂O₃ | 0 to 11.5 |
| Ga₂O₃ | 0 to 5.0 |
| In₂O₃ | 0 to 5.0 |

In a particularly preferred embodiment, the glass ceramic comprises 0.1 to 6.0, preferably 1.0 to 5.0, more preferably 1.5 to 4.0, and most preferably 1.5 to 3.0 wt.-% Al₂O₃.

Furthermore, a glass ceramic comprising 0 to 9.0 and particularly preferably 0 to 7.0 wt.-% oxide of tetravalent elements Me^{IV}O₂ selected from the group of ZrO₂, TiO₂, GeO₂ and mixtures thereof is preferred.

Particularly preferably, the glass ceramic comprises at least one and, in particular, all of the following oxides of tetravalent elements Me^{IV}O₂ in the amounts indicated:

| Component | Wt.-% |
|---|---|
| ZrO₂ | 0 to 9.0 |
| TiO₂ | 0 to 6.0 |
| GeO₂ | 0 to 4.0 |

In another preferred embodiment, the glass ceramic comprises 0 to 10.0 and preferably 0 to 8.0 wt.-% oxide of pentavalent elements Me^{V}₂O₅ selected from the group consisting of Ta₂O₅ and Nb₂O₅ and mixtures thereof.

Particularly preferably, the glass ceramic comprises at least one and, in particular, all of the following oxides of pentavalent elements Me^{V}₂O₅ in the amounts indicated:

| Component | Wt.-% |
|---|---|
| Ta₂O₅ | 0 to 8.0 |
| Nb₂O₅ | 0 to 10.0 |

It is also preferred that the glass ceramic according to the invention comprises less than 3.0, preferably less than 2.0, more preferably less than 1.0, and most preferably less than 0.1 wt.-% P₂O₅. In a further preferred embodiment, the glass ceramic is substantially free of P₂O₅.

In another embodiment, the glass ceramic comprises 0 to 7.0 and preferably 0 to 6.0 wt.-% oxide of hexavalent element Me^{VI}O₃ selected from the group consisting of WO₃, MoO₃ and mixtures thereof.

Particularly preferably, the glass ceramic comprises at least one, and in particular all, of the following oxides Me^{VI}O₃ in the amounts indicated:

| Component | Wt.-% |
|---|---|
| WO₃ | 0 to 4.5 |
| MoO₃ | 0 to 5.5 |

In a further embodiment, the glass ceramic according to the invention comprises 0 to 1.0 and in particular 0 to 0.5 wt.-% fluorine.

Particularly preferred is a glass ceramic comprising at least one, and preferably all, of the following components in the amounts indicated:

| Component | Wt.-% |
|---|---|
| SiO₂ | 65.0 to 89.0 |
| Li₂O | 10.0 to 21.0 |
| Tin, calculated | 0.01 to 4.5 |
| as SnO₂, | |
| P₂O₅ | less than 3.0 |
| Me^{I}₂O | 0 to 7.0 |
| Me^{II}O | 0 to 15.0 |
| Me^{III}₂O₃ | 0 to 12.0 |
| Me^{IV}O₂ | 0 to 9.0 |
| Me^{V}₂O₅ | 0 to 10.0 |
| Me^{VI}O₃ | 0 to 7.0 |
| Fluorine | 0 to 1.0, |

where Me^{I}₂O, Me^{II}O, Me^{III}₂O₃, Me^{IV}O₂, Me^{V}₂O₅ and Me^{VI}O₃ have the meanings given above.

In another particularly preferred embodiment, the glass ceramic comprises at least one, and preferably all, of the following components in the amounts indicated:

| Component | Wt.-% |
|---|---|
| SiO₂ | 68.0 to 83.0 |
| Li₂O | 11.0 to 20.0 |
| Tin, calculated | 0.03 to 3.0 |
| as SnO₂, | |
| P₂O₅ | less than 2.0 |
| K₂O | 0 to 6.0 |
| Na₂O | 0 to 6.0 |
| Rb₂O | 0 to 5.0 |
| Cs₂O | 0 to 4.0 |
| CaO | 0 to 10.0 |
| MgO | 0 to 8.0 |
| SrO | 0 to 15.0 |
| ZnO | 0 to 12.0 |
| Al₂O₃ | 0 to 6.0 |
| B₂O₃ | 0 to 5.0 |
| Y₂O₃ | 0 to 8.5 |
| La₂O₃ | 0 to 11.5 |
| Ga₂O₃ | 0 to 5.0 |
| In₂O₃ | 0 to 5.0 |
| ZrO₂ | 0 to 9.0 |
| TiO₂ | 0 to 6.0 |
| GeO₂ | 0 to 4.0 |
| Ta₂O₅ | 0 to 8.0 |
| Nb₂O₅ | 0 to 10.0 |
| WO₃ | 0 to 4.5 |
| MoO₃ | 0 to 5.5 |
| Fluorine | 0 to 0.5. |

Some of the above components may serve as coloring agents and/or fluorescent agents. The glass ceramic according to the invention may furthermore comprise further coloring agents and/or fluorescent agents. These may in particular be selected from further inorganic pigments and/or oxides of d and f elements, such as the oxides of Mn, Fe, Co, Pr, Nd, Tb, Er, Dy, Eu and Yb, or metals, preferably Ag, Cu and Au.

In a preferred embodiment of the glass ceramic, the molar ratio of SiO₂ to Li₂O is in the range of 1.5 to 4.0, preferably 1.7 to 3.5, and more preferably 2.0 to 3.0.

It is further preferred that the glass ceramic according to the invention comprises lithium disilicate or lithium metasilicate as the main crystal phase and, in particular, lithium disilicate as the main crystal phase.

The term “main crystal phase” refers to the crystal phase which has the highest weight proportion of all crystal phases present in the glass ceramic. The amounts of the crystal phases are determined in particular by the Rietveld method. A suitable procedure for the quantitative analysis of the crystal phases by means of the Rietveld method is described, for example, in the dissertation by M. Dittmer “Gläser and Glaskeramiken im System MgO—Al₂O₃-SiO₂ mit ZrO₂ als Keimbildner”, University of Jena 2011.

It is preferred that the glass ceramic according to the invention comprises at least 1.0 wt.-%, preferably at least 1.5 wt.-% and particularly preferably at least 2.0 wt.-% lithium metasilicate crystals. Particularly preferably, the glass ceramic according to the invention comprises 1.0 to 50.0 wt.-%, preferably 1.5 to 45.0 wt.-% and especially preferably 2.0 to 40.0 wt.-% lithium metasilicate crystals.

In another embodiment, it is preferred that the glass ceramic according to the invention comprises at least 50.0 wt.-%, preferably at least 55.0 wt.-% and particularly preferably at least 60.0 wt.-% lithium disilicate crystals. Particularly preferably, the glass ceramic according to the invention comprises 50.0 to 90.0 wt.-%, preferably 55.0 to 85.0 wt.-% and especially preferably 60.0 to 80.0 wt.-% lithium disilicate crystals.

The glass ceramic according to the invention is characterized by particularly good mechanical and optical properties and it can be formed by heat treatment of a corresponding starting glass or a corresponding starting glass with nuclei. These materials can therefore serve as precursors for the glass ceramic according to the invention.

The type and, in particular, the amount of crystal phases formed can be controlled by the composition of the starting glass as well as the heat treatment applied to produce the glass ceramic from the starting glass. The examples illustrate this by varying the composition of the starting glass and the heat treatment applied.

The glass ceramic has a high biaxial fracture strength of preferably at least 150 MPa and particularly preferably at least 250 MPa. The biaxial fracture strength was determined in accordance with ISO 6872 (2008) (piston-on-three-balls test).

The glass ceramic also has a high fracture toughness of preferably at least 1.5 MPa·m^{0.5}, particularly preferably at least 2.0 MPa·m^{0.5} and most preferably at least 2.5 MPa·m^{0.5}. The fracture toughness was determined according to ISO 6872 (2015) (SEVNB method).

Further, the glass ceramic has a high chemical stability measured as acid solubility according to ISO 6872 (2015) of preferably less than 100 g/cm².

The particular combination of properties present in the glass ceramic according to the invention even allows it to be used as a dental material and, in particular, as a material for producing dental restorations.

The invention also relates to precursors of corresponding composition from which the glass ceramic according to the invention can be produced by heat treatment. These precursors are a correspondingly composed starting glass and a correspondingly composed starting glass with nuclei. The term “corresponding composition” means that these precursors comprise the same components in the same amounts as the glass ceramic, the components being calculated as oxides as is usual for glasses and glass ceramics, with the exception of fluorine.

The invention therefore also relates to a starting glass comprising the components of the glass ceramic according to the invention.

The starting glass according to the invention therefore comprises, in particular, suitable amounts of SiO₂, Li₂O and tin, which are required to form the glass ceramic according to the invention. Further, the starting glass may also comprise other components as indicated above for the glass ceramic according to the invention. All such embodiments are preferred for the components of the starting glass that are also indicated as preferred for the components of the glass ceramic according to the invention.

Particularly preferably, the starting glass is in the form of a monolithic blank obtained by casting a melt of the starting glass into a mold.

The invention also relates to such a starting glass comprising nuclei for the crystallization of lithium silicate, in particular lithium metasilicate and/or lithium disilicate.

The starting glass is produced in particular by melting a mixture of suitable starting materials, such as carbonates, oxides and halides, at temperatures of in particular about 1500 to 1800° C. for 0.5 to 4 h. In particular, SnO or SnO₂ can be used as the starting material for tin. The melt can then be poured into water to produce a frit. To achieve a particularly high homogeneity, the glass frit obtained is again melted.

The melt can then be poured into molds to produce blanks of the starting glass, so-called solid glass blanks or monolithic blanks.

By heat treatment of the starting glass, the further precursor starting glass with nuclei can first be produced. The lithium silicate glass ceramic according to the invention can then be produced by heat treatment of this further precursor. Alternatively, the glass ceramic according to the invention can be famed by heat treatment of the starting glass.

It is preferred to subject the starting glass to a heat treatment at a temperature of 400 to 600° C., especially 430 to 550° C. and particularly preferably 440 to 520° C. for a duration of preferably 5 to 120 min, especially 10 to 60 min, to produce the starting glass with nuclei for the crystallization of lithium silicate.

It is further preferred to subject the starting glass or the starting glass with nuclei to a heat treatment at a temperature of 800 to 1050° C., preferably 850 to 1020° C., for a duration of in particular 5 seconds to 120 min, preferably 1 min to 100 min, more preferably 5 min to 60 min and further preferred 10 min to 30 min, in order to produce the glass ceramic according to the invention.

The invention therefore also relates to a process for producing the glass ceramic according to the invention, in which the starting glass or the starting glass with nuclei is subjected to at least one heat treatment in the range from 800 to 1050° C., preferably 850 to 1020° C., for a duration of in particular 5 seconds to 120 min, preferably 1 min to 100 min, more preferably 5 min to 60 min and further preferred 10 min to 30 min.

The at least one heat treatment carried out in the process according to the invention can also be carried out in the course of hot pressing, in particular of a solid glass blank, or sintering, in particular of a powder, of the starting glass according to the invention or of the starting glass according to the invention with nuclei.

In a further preferred embodiment, the starting glass or the starting glass with nuclei can first be subjected to a heat treatment at a temperature of 550 to 800° C., preferably 600 to 800° C., for a duration of in particular 5 seconds to 120 min, preferably 1 min to 100 min, particularly preferably 5 min to 60 min and further preferred 10 min to 30 min, in order to produce the glass ceramic according to the invention with lithium metasilicate as the main crystal phase.

The glass ceramic according to the invention with lithium metasilicate as the main crystal phase can then be subjected to a further heat treatment to convert lithium metasilicate crystals into lithium disilicate crystals and, in particular, to form the glass ceramic according to the invention with lithium disilicate as the main crystal phase. Preferably, the glass ceramic is subjected to a further heat treatment at a temperature of 800 to 1050° C., preferably 850 to 1020° C. and particularly preferably 900 to 1020° C., in particular for a duration of 5 seconds to 120 min, preferably 1 min to 100 min, particularly preferably 1 min to 60 min, further preferred 5 to 30 min and most preferably 5 to 20 min.

The appropriate conditions for a given glass ceramic can be determined, for example, by performing X-ray diffraction analyses at different temperatures.

The glass ceramics according to the invention and the glasses according to the invention are present in particular in the form of powders, granules or blanks in any shape and size, e.g. monolithic blanks, such as platelets, cuboids or cylinders, or powder compacts, in unsintered, partially sintered or densely sintered form. In these forms, they can be easily further processed, e.g. into dental restorations. However, they can also be in the form of dental restorations, such as inlays, onlays, crowns, veneers, facets or abutments.

Dental restorations, such as bridges, inlays, onlays, crowns, veneers, facets or abutments, can be produced from the glass ceramics according to the invention and the glasses according to the invention. The invention therefore also relates to their use in producing dental restorations. In this context, it is preferred that the glass ceramic or the glass is given the shape of the desired dental restoration by pressing and in particular by machining.

The pressing is usually carried out under elevated pressure and temperature. It is preferred that the pressing is carried out at a temperature of 700 to 1200° C. It is further preferred that the pressing be carried out at a pressure of 2 to 10 bar. During pressing, the desired change in shape is achieved by viscous flow of the material used. The starting glass according to the invention, the starting glass with nuclei according to the invention and the glass ceramic according to the invention can be used for the pressing. In particular, the glass and glass ceramics according to the invention can be used in the form of blanks of any shape and size.

Machining is usually carried out by material-removing processes and in particular by milling and/or grinding. It is particularly preferred that the machining is carried out in a CAD/CAM process. The starting glass according to the invention, the starting glass with nuclei according to the invention and the glass ceramic according to the invention can be used for the machining. Preferably, the starting glass with nuclei or the glass ceramic according to the invention with lithium metasilicate as the main crystal phase are used. In this context, the glasses and glass ceramics according to the invention can be used in particular in the form of blanks.

Due to the above-described properties of the glass ceramics according to the invention and the glasses according to the invention, they are particularly suitable for use in dentistry. It is therefore also an object of the invention to use the glass ceramics according to the invention or the glasses according to the invention as dental material and preferably for producing dental restorations, such as bridges, inlays, onlays, veneers, abutments, partial crowns, crowns or facets.

The invention thus also relates to a process for producing a dental restoration, in particular a bridge, inlay, onlay, veneer, abutment, partial crown, crown or facet, in which the glass ceramic or glass according to the invention is given the shape of the desired dental restoration by pressing or by machining, in particular in a CAD/CAM process.

The invention is explained in more detail below by means of nonlimiting examples.

### EXAMPLES

### Examples 1 to 49—Composition and Crystal Phases

A total of 49 glasses and glass ceramics according to the invention with the composition indicated in Table I were produced via melting of corresponding starting materials to produce starting glasses and their subsequent heat treatment for controlled crystallization.

The applied heat treatments as well as properties of the obtained glass ceramics are also given in Table I. The following meanings apply
T_{g} Glass transition temperature determined by DSC
T_{S} and t_{S} Applied temperature and time for melting of the starting glass
T_{Kb} and t_{Kb} Applied temperature and time for nucleation of starting glass
T_{C1} and t_{C1} Applied temperature and time for first crystallization
T_{C2} and t_{C2} Applied temperature and time for second crystallization
K_{IC} Fracture toughness measured according to ISO 6872 (2015) (SEVNB method)
Chem. Stability Measured as loss in mass according to ISO 6872 (2015)
σ_{Biax} Biaxial fracture strength measured according to ISO 6872 (2015) (piston-on-three-balls test).

In the examples, starting glasses with the compositions given in Table I were first melted on a 100 to 200 g scale from common raw materials at temperature T_{S} for duration t_{S}, with very good melting being possible without the formation of bubbles or streaks. Glass frits were prepared by pouring the starting glasses into water, which optionally were subsequently melted a second time at temperature T_{S} for duration t_{S} for homogenization. The resulting melts of the starting glass were then poured into a graphite mold to produce monolithic glass blocks.

A first heat treatment of the obtained glass blocks at temperature T_{Kb} for duration t_{Kb} resulted in relaxation of the glasses and formation of glasses with nuclei. These nucleated glasses crystallized by further heat treatment at temperature T_{C1} for duration t_{C1} to form glass ceramics with lithium metasilicate or lithium disilicate as the main crystalline phase, as determined by X-ray diffraction studies at room temperature. In some cases, further heat treatment at temperature T_{C2} for duration t_{C2} was subsequently carried out, resulting in glass ceramics with lithium disilicate as the main crystalline phase.

In Examples 9 and 38, glass frits were produced by pouring the starting glasses into water. These frits were crushed, sieved and subsequently sintered at the temperature and time indicated in Table I.

The amounts of the crystal phases were determined by X-ray diffraction. For this purpose, powders of the respective glass ceramics were prepared by grinding and sieving (<45 μm) and admixed with Al₂O₃ (Alfa Aesar, product no. 42571) as internal standard in a ratio of 80 wt.-% glass ceramic to 20 wt.-% Al₂O₃. The mixture was slurried with acetone to achieve the best possible mixing. The mixture was then dried at about 80° C. A diffractogram was then recorded using a Bruker D8 Advance diffractometer in the range 10 to 100° 2θ using CuKα radiation and a step size of 0.014° 2θ. This diffractogram was then analyzed using Bruker's TOPAS 5.0 software using the Rietveld method. By comparing the intensities of the peaks with those of Al₂O₃, the phase fractions were determined.

To determine the biaxial fracture strengths according to ISO 6872 (2015) (piston-on-three-balls test), holders were bonded to blocks of the relaxed and nucleated glasses, and these blocks were subsequently machined using a CAD/CAM grinding unit (Sirona InLab). The grinding process was performed using diamond-coated grinding tools. The resulting platelets were subjected to the heat treatment indicated in the table, i.e., first crystallization and, if necessary, second crystallization, and then the crystallized platelets were polished to a thickness of 1.2±0.2 mm using diamond wheels. The biaxial fracture strength was determined on the specimens prepared in this way.

High biaxial fracture strengths ranging from more than 179 to 524 MPa were determined for the glass ceramics produced.

Fracture toughnesses were determined according to ISO 6872 (2015) (SEVNB method), and high fracture toughnesses in the range of 2.6 to 3.1 MPa·m^{0.5} were determined for the produced glass ceramics.

Chemical stability testing was performed according to ISO 6872 (2015), and the glass ceramics produced showed an acid solubility of less than 100 g/cm².

Dental crowns were fabricated from the produced glasses and glass ceramics by CAD/CAM-supported machining, and these crowns were optionally subjected to a final crystallization under the conditions indicated in Table I.

### Example 50—Comparative

In this example a starting glass comprising no tin was prepared via melting of corresponding starting materials and this glass was subsequently heat treated to effect its crystallization.

The manufacturing process was the same as the process described for the preparation of Examples 1 to 49. The composition used, the applied heat treatments as well as properties of the obtained glass ceramic are also given in Table I.

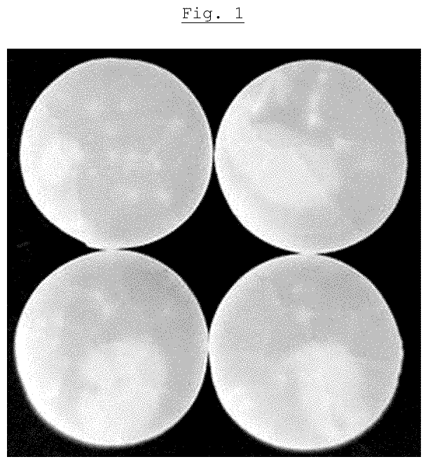

FIG. 1 shows four platelets of the obtained glass ceramic. It can be seen that in these samples, which contain no tin, cracking occurs due to uncontrolled crystal growth.

TABLE I

| | Example no. | | | | |
|---|---|---|---|---|---|
| | 1 | 2 | 3 | 4 | 5 |
| Composition | Wt.-% | Wt.-% | Wt.-% | Wt.-% | Wt.-% |
| SiO₂ | 79.70 | 79.70 | 79.70 | 79.70 | 79.70 |
| Li₂O | 16.51 | 16.51 | 16.51 | 16.51 | 16.51 |
| SnO₂ | 0.03 | 0.03 * | 0.03 * | 0.03 * | 0.03 * |
| Na₂O | | | | | |
| K₂O | 1.81 | 1.81 | 1.81 | 1.81 | 1.81 |
| Al₂O₃ | 1.95 | 1.95 | 1.95 | 1.95 | 1.95 |
| Σ | 100.00 | 100.00 | 100.00 | 100.00 | 100.00 |
| T_{g}/° C. | 461.6° C. | 456.3 | 453.9 | 453.9 | 453.1 |
| Tₛ/° C. | 1650 + 1650 | 1500 + 1500 | 1650 + 1650 | 1650 + 1650 | 1750 + 1750 |
| tₛ/min. | 60 + 60 | 60 + 60 | 60 + 60 | 60 + 60 | 45 + 45 |
| T_{Kb}/° C. | 480 | 480 | 480 | 480 | 480 |
| t_{Kb}/min | 10 | 10 | 10 | 10 | 10 |
| T_{C1}/° C. | 950 | 950 | 620 | 950 | 950 |
| t_{C1}/min. | 10 | 10 | 10 | 10 | 10 |
| T_{C2}/° C. | | | | | |
| t_{C2}/min. | | | | | |
| Main crystal | Li₂Si₂O₅ | Li₂Si₂O₅ | Li₂SiO₃ | Li₂Si₂O₅ | Li₂Si₂O₅ |
| phase (wt.-%) | (76.0) | | | | |
| Other crystal | | Li₂SiO₃ | Li₂Si₂O₅ | | |
| phases (wt.-%) | | Quartz | | | |
| K_{IC} (MPa*m^{0.5}) | 2.9 ± 0.2 | | | | |
| Chem. stability | | | | | |
| (μg/cm²) | | | | | |
| σ_{Biax} (MPa) | 358 ± 33 | | | | |
| | Example no. | | | | |
| | 6 | 7 | 8 | 9 | 10 |
| Composition | Wt.-% | Wt.-% | Wt.-% | Wt.-% | Wt.-% |
| SiO₂ | 79.26 | 79.49 | 79.26 | 79.26 | 74.45 |
| Li₂O | 16.41 | 16.47 | 16.41 | 16.41 | 15.41 |
| SnO₂ | 0.58 * | 0.29 | 0.58 | 0.58 | 0.57 |
| Na₂O | | | | | 5.86 |
| K₂O | 1.80 | 1.80 | 1.80 | 1.80 | 1.78 |
| Al₂O₃ | 1.95 | 1.95 | 1.95 | 1.95 | 1.93 |
| Σ | 100.00 | 100.00 | 100.00 | 100.00 | 100.00 |
| T_{g}/° C. | 452.9 | 463.8 | 460.1 | 460.1 | 431.7 |
| Tₛ/° C. | 1500 + 1500 | 1650 + 1650 | 1650 + 1650 | 1650 | 1650 + 1650 |
| tₛ/min. | 60 + 80 | 60 + 60 | 60 + 60 | 60 | 60 + 60 |
| T_{Kb}/° C. | 480 | 480 | 480 | 480 | 480 |
| t_{Kb}/min | 10 | 10 | 10 | 10 | 10 |
| T_{C1}/° C. | 950 | 950 | 950 | 1000 | 900 |
| | | | | sintered | |
| t_{C1}/min. | 10 | 10 | 10 | 10 sintered | 10 |
| T_{C2}/° C. | | | | | |
| t_{C2}/min. | | | | | |
| Main crystal | Li₂Si₂O₅ | Li₂Si₂O₅ | Li₂Si₂O₅ | Li₂Si₂O₅ | Li₂Si₂O₅ |
| phase (wt.-%) | | (75.4) | (72.8) | | |
| Other crystal | | | | | Li₂SiO₃ |
| phases (wt.-%) | | | | | |
| K_{IC} (MPa*m^{0.5}) | | 2.9 ± 0.1 | 3.0 ± 0.2 | 2.9 ± 0.1 | |
| Chem. stability | | | 10 | | |
| (μg/cm²) | | | | | |
| σ_{Biax} (MPa) | | 379 ± 32 | 337 ± 33 | 276 ± 14 | |

| | Example no. | | | | | |
|---|---|---|---|---|---|---|
| | 11 | 12 | 13 | 14 | 15 | 16 |
| Composition | Wt.-% | Wt.-% | Wt.-% | Wt.-% | Wt.-% | Wt.-% |
| SiO₂ | 77.17 | 74.00 | 72.24 | 67.39 | 69.57 | 76.03 |
| Li₂O | 15.97 | 15.33 | 14.96 | 13.95 | 14.41 | 15.74 |
| SnO₂ | 0.56 | 0.59 | 0.57 | 0.53 | 0.55 | 0.57 |
| K₂O | 1.76 | 1.83 | 1.79 | 1.67 | 1.72 | 1.78 |
| Cs₂O | 2.63 | | | | | |
| MgO | | 6.27 | | | | |
| CaO | | | 8.51 | | | |
| SrO | | | | 14.66 | | |
| ZnO | | | | | 11.89 | |
| Al₂O₃ | 1.91 | 1.98 | 1.93 | 1.80 | 1.86 | 1.93 |
| B₂O₃ | | | | | | 3.95 |
| La₂O₃ | | | | | | |
| Y₂O₃ | | | | | | |
| Er₂O₃ | | | | | | |
| Dy₂O₃ | | | | | | |
| Σ | 100.00 | 100.00 | 100.00 | 100.00 | 100.00 | 100.00 |
| T_{g}/° C. | 457.1 | 454.5 | 461.9 | 449.3 | 447.9 | 463.7 |
| Tₛ/° C. | 1650 + 1650 | 1650 + 1650 | 1650 + 1650 | 1650 + 1650 | 1650 + 1650 | 1650 + 1650 |
| tₛ/min. | 60 + 60 | 60 + 60 | 60 + 60 | 60 + 60 | 60 + 60 | 60 + 60 |
| T_{Kb}/° C. | 480 | 480 | 480 | 480 | 480 | 480 |
| t_{Kb}/min | 10 | 10 | 10 | 10 | 10 | 10 |
| T_{C1}/° C. | 950 | 850 | 850 | 890 | 900 | 900 |
| t_{C1}/min. | 10 | 10 | 10 | 10 | 10 | 10 |
| T_{C2}/° C. | | | | | | |
| t_{C2}/min. | | | | | | |
| Main crystal | Li₂Si₂O₅ | Li₂Si₂O₅ | Li₂Si₂O₅ | Li₂Si₂O₅ | Li₂Si₂O₅ | Li₂Si₂O₅ |
| phase (wt.-%) | | | | | | |
| Other crystal | Li₂SiO₃ | Li₂SiO₃ | Li₂SiO₃ | Li₂Si₂O₃ | Li₂SiO₃ | Li₂SiO₃ |
| phases (wt.-%) | | MgSiO₃ | CaSiO₃ | | Quartz | Quartz |
| | | (enstatite) | (diopside) | | Li₂ZnSiO₄ | |
| K_{IC} (MPa*m^{0.5}) | | | | | | |
| Chem. stability | | | | | | |
| (μg/cm²) | | | | | | |
| σ_{Biax} (MPa) | | | | | | |

| | Example no. | | | |
|---|---|---|---|---|
| | 17 | 18 | 19 | 20 |
| Composition | Wt.-% | Wt.-% | Wt.-% | Wt.-% |
| SiO₂ | 70.27 | 72.78 | 78.34 | 78.42 |
| Li₂O | 14.55 | 15.07 | 16.22 | 16.24 |
| SnO₂ | 0.52 | 0.54 | 0.57 | 0.57 |
| K₂O | 1.63 | 1.69 | 1.78 | 1.78 |
| Cs₂O | | | | |
| MgO | | | | |
| CaO | | | | |
| SrO | | | | |
| ZnO | | | | |
| Al₂O₃ | 1.76 | 1.83 | 1.93 | 1.93 |
| B₂O₃ | | | | |
| La₂O₃ | 11.27 | | | |
| Y₂O₃ | | 8.09 | | |
| Er₂O₃ | | | 1.16 | |
| Dy₂O₃ | | | | 1.06 |
| Σ | 100.00 | 100.00 | 100.00 | 100.00 |
| T_{g}/° C. | 468.2 | 482.4 | 457.6 | 460.9 |
| Tₛ/° C. | 1650 + 1650 | 1650 + 1650 | 1650 + 1650 | 1650 + 1650 |
| tₛ/min. | 60 + 60 | 60 + 60 | 60 + 60 | 60 + 60 |
| T_{Kb}/° C. | 480 | 480 | 480 | 480 |
| t_{Kb}/min | 10 | 10 | 10 | 10 |
| T_{C1}/° C. | 930 | 930 | 970 | 950 |
| t_{C1}/min. | 10 | 10 | 10 | 10 |
| T_{C2}/° C. | | | | |
| t_{C2}/min. | | | | |
| Main crystal | Li₂Si₂O₅ | Li₂Si₂O₅ | Li₂Si₂O₅ | Li₂Si₂O₅ |
| phase (wt.-%) | | (50.9) | (67.1) | |
| Other crystal | Li₂SiO₃ | Li₂SiO₃ (2.1) | | |
| phases (wt.-%) | Li₂Si₂O₇ | Quartz (0.2) | | |
| K_{IC} (MPa*m^{0.5}) | | 2.7 ± 0.1 | 2.9 ± 0.3 | 2.8 ± 0.1 |
| Chem. stability | | | | |
| (μg/cm²) | | | | |
| σ_{Biax} (MPa) | | 286 ± 25 | 179 ± 31 | 426 ± 21 |

| | Example no. | | | | |
|---|---|---|---|---|---|
| | 21 | 22 | 23 | 24 | 25 |
| Composition | Wt.-% | Wt.-% | Wt.-% | Wt.-% | Wt.-% |
| SiO₂ | 78.42 | 72.05 | 74.40 | 76.10 | 71.76 |
| Li₂O | 16.24 | 14.92 | 15.40 | 15.75 | 14.86 |
| SnO₂ | 0.57 | 0.55 | 0.56 | 0.56 | 0.53 |
| K₂O | 1.78 | 1.71 | 1.76 | 1.76 | 1.66 |
| Al₂O₃ | 1.93 | 1.85 | 1.91 | 1.91 | 1.80 |
| Dy₂O₃ | 1.06 | | | | |
| ZrO₂ | | 8.92 | | | |
| TiO₂ | | | 5.97 | | |
| GeO₂ | | | | 3.92 | |
| Nb₂O₅ | | | | | 9.39 |
| Ta₂O₅ | | | | | |
| P₂O₅ | | | | | |
| MoO₃ | | | | | |
| WO₃ | | | | | |
| F | | | | | |
| Σ | 100.00 | 100.00 | 100.00 | 100.00 | 100.00 |
| T_{g}/° C. | 460.9 | 493.4 | 465.7 | 456.3 | 476.3 |
| Tₛ/° C. | 1650 + 1650 | 1650 + 1650 | 1650 + 1650 | 1650 + 1650 | 1650 + 1650 |
| tₛ/min. | 60 + 60 | 60 + 60 | 60 + 60 | 60 + 60 | 60 + 60 |
| T_{Kb}/° C. | 480 | 480 | 480 | 480 | 480 |
| t_{Kb}/min | 10 | 10 | 10 | 10 | 10 |
| T_{C1}/° C. | 970 | 940 | 900 | 950 | 920 |
| t_{C1}/min. | 10 | 10 | 10 | 10 | 10 |
| T_{C2}/° C. | | | | | |
| t_{C2}/min. | | | | | |
| Main crystal | Li₂Si₂O₅ | Li₂Si₂O₅ | Li₂Si₂O₅ | Li₂Si₂O₅ | Li₂Si₂O₅ |
| phase (wt.-%) | (69.5) | | | | |
| Other crystal | | Li₂SiO₃ | Li₂Si₂O₅ | | Li₂SiO₃ |
| phases (wt.-%) | | | Quartz | | Quartz |
| | | | TiO₂ | | Li_{0.938}Nb_{0.012}(NbO₃) |
| K_{IC} (MPa*m^{0.5}) | | 2.6 ± 0.1 | | | |

| | Example no. | | | | |
|---|---|---|---|---|---|
| | 26 | 27 | 28 | 29 | 30 |
| Composition | Wt.-% | Wt.-% | Wt.-% | Wt.-% | Wt.-% |
| SiO₂ | 73.00 | 77.11 | 74.99 | 75.84 | 78.63 |
| Li₂O | 15.12 | 15.97 | 15.52 | 15.70 | 16.27 |
| SnO₂ | 0.54 | 0.57 | 0.56 | 0.56 | 0.58 * |
| K₂O | 1.67 | 1.77 | 1.74 | 1.74 | 1.82 |
| Al₂O₃ | 1.81 | 1.91 | 1.88 | 1.88 | 1.97 |
| Dy₂O₃ | | | | | |
| ZrO₂ | | | | | |
| TiO₂ | | | | | |
| GeO₂ | | | | | |
| Nb₂O₅ | | | | | |
| Ta₂O₅ | 7.86 | | | | |
| P₂O₅ | | 2.67 | | | |
| MoO₃ | | | 5.31 | | |
| WO₃ | | | | 4.28 | |
| F | | | | | 0.73 |
| Σ | 100.00 | 100.00 | 100.00 | 100.00 | 100.00 |
| T_{g}/° C. | 468.7 | 463.1 | 455.8 | 465.9 | 438.6 |
| Tₛ/° C. | 1650 + 1650 | 1650 + 1650 | 1650 + 1650 | 1650 + 1650 | 1650 + 1650 |
| tₛ/min. | 60 + 60 | 60 + 60 | 60 + 60 | 60 + 60 | 60 + 60 |
| T_{Kb}/° C. | 480 | 480 | 480 | 480 | 480 |
| t_{Kb}/min | 10 | 10 | 10 | 10 | 10 |
| T_{C1}/° C. | 890 | 950 | 930 | 960 | 950 |
| t_{C1}/min. | 10 | 10 | 10 | 10 | 10 |
| T_{C2}/° C. | | | | | |
| t_{C2}/min. | | | | | |
| Main crystal | Li₂Si₂O₅ | Li₂Si₂O₅ | Li₂Si₂O₅ | Li₂Si₂O₅ | Li₂Si₂O₅ |
| phase (wt.-%) | | (61.2) | | | |
| Other crystal | Li₂SiO₃ | Li₃PO₄ (3.3) | Li₂SiO₃ | Li₂WO₄ | Tridymite |
| phases (wt.-%) | Quartz | Cristobalite (4.6) | Quartz | | Quartz |
| | | | Cristobalite | | |
| | | | Li₂MoO₄ | | |
| K_{IC} (MPa*m^{0.5}) | | | | | |

| | Example no. | | | | |
|---|---|---|---|---|---|
| | 31 | 32 | 33 | 34 | 35 |
| Composition | Wt.-% | Wt.-% | Wt.-% | Wt.-% | Wt.-% |
| SiO₂ | 79.16 | 79.2489 | 79.04 | 79.04 | 78.80 |
| Li₂O | 16.40 | 16.42 | 16.36 | 16.36 | 16.32 |
| SnO₂ | 0.58 | 0.58 | 0.86 | 0.86 | 1.15 |
| K₂O | 1.80 | 1.80 | 1.80 | 1.80 | 1.79 |
| Al₂O₃ | 1.95 | 1.95 | 1.94 | 1.94 | 1.94 |
| AgCl | 0.11 | 0.0011 | | | |
| Σ | 100.00 | 100.0000 | 100.00 | 100.00 | 100.00 |
| T_{g}/° C. | 458.8 | 454.7 | 460.7 | 460.7 | 462.6 |
| Tₛ/° C. | 1650 + 1650 | 1650 + 1650 | 1650 + 1650 | 1650 + 1650 | 1650 + 1650 |
| tₛ/min. | 60 + 60 | 60 + 60 | 60 + 60 | 60 + 60 | 60 + 60 |
| T_{Kb}/° C. | 480 | 480 | 480 | 480 | 480 |
| t_{Kb}/min | 10 | 10 | 10 | 10 | 10 |
| T_{C1}/° C. | 950 | 950 | 950 | 630 | 950 |
| t_{C1}/min. | 10 | 10 | 10 | 10 | 10 |
| T_{C2}/° C. | | | | 950 | |
| t_{C2}/min. | | | | 10 | |
| Main crystal | Li₂Si₂O₅ | Li₂Si₂O₅ | Li₂Si₂O₅ | Li₂Si₂O₅ | Li₂Si₂O₅ |
| phase (wt.-%) | | | | (73.4) | |
| Other crystal | Li₂SiO₃ | Li₂SiO₃ | | | |
| phases (wt.-%) | Quartz | Quartz | | | |
| K_{IC} (MPa*m^{0.5}) | | 3.1 ± 0.1 | | | |
| Chem. stability | | | | | |
| (μg/cm²) | | | | | |
| σ_{Biax} (MPa) | | 195 ± 18 | | 433 ± 46 | |

| | Example no. | | | | |
|---|---|---|---|---|---|
| | 36 | 37 | 38 | 39 | 40 |
| Composition | Wt.-% | Wt.-% | Wt.-% | Wt.-% | Wt.-% |
| SiO₂ | 78.80 | 78.57 | 78.57 | 76.78 | 82.40 |
| Li₂O | 16.32 | 16.27 | 16.27 | 19.09 | 13.66 |
| SnO₂ | 1.15 | 1.43 | 1.43 | 0.29 | 0.28 |
| K₂O | 1.79 | 1.79 | 1.79 | 1.84 | 1.76 |
| Al₂O₃ | 1.94 | 1.94 | 1.94 | 2.00 | 1.90 |
| AgCl | | | | | |
| Σ | 100.00 | 100.00 | 100.00 | 100.00 | 100.00 |
| T_{g}/° C. | 462.6 | 460.7 | 460.7 | 451.8 | 465.6 |
| Tₛ/° C. | 1650 + 1650 | 1650 + 1650 | 1650 + 1650 | 1650 + 1650 | 1650 + 1650 |
| tₛ/min. | 60 + 60 | 60 + 60 | 60 + 60 | 60 + 60 | 60 + 60 |
| T_{Kb}/° C. | 480 | 480 | 480 | 480 | 480 |
| t_{Kb}/min | 10 | 10 | 10 | 10 | 10 |
| T_{C1}/° C. | 630 | 950 | 980 | 950 | 950 |
| | | | sintered | | |
| t_{C1}/min. | 10 | 10 | 10 sintered | 10 | 10 |
| T_{C2}/° C. | 950 | | | | |
| t_{C2}/min. | 10 | | | | |
| Main crystal | Li₂Si₂O₅ | Li₂Si₂O₅ | Li₂Si₂O₅ | Li₂Si₂O₅ | Li₂Si₂O₅ |
| phase (wt.-%) | (72.8) | (72.6 ) | | | |
| Other crystal | | | | Li₂SiO₃ | Quartz |
| phases (wt.-%) | | | | | cristobalite |
| K_{IC} (MPa*m^{0.5}) | 2.8 ± 0.1 | 2.9 ± 0.1 | | | |
| Chem. stability | | | | | |
| (μg/cm²) | | | | | |
| σ_{Biax} (MPa) | 490 ± 40 | 399 ± 34 | | | |

| | Example no. | | | | | |
|---|---|---|---|---|---|---|
| | 41 | 42 | 43 | 44 | 45 | 46 |
| Composition | Wt.-% | Wt.-% | Wt.-% | Wt.-% | Wt.-% | Wt.-% |
| SiO₂ | 84.15 | 76.17 | 78.00 | 77.43 | 81.10 | 78.02 |
| Li₂O | 11.96 | 15.81 | 16.15 | 16.04 | 16.79 | 16.17 |
| SnO₂ | 0.28 | 0.28 | 2.14 | 2.84 | 0.29 | 0.29 |
| K₂O | 1.73 | 4.12 | 1.78 | 1.77 | 1.82 | 3.58 |
| Al₂O₃ | 1.88 | 3.62 | 1.93 | 1.92 | | 1.94 |
| P₂O₅ | | | | | | |
| Σ | 100.00 | 100.00 | 100.00 | 100.00 | 100.00 | 100.00 |
| T_{g}/° C. | 470.6 | 455.7 | 463.1 | 461.3 | 460.2 | 446 |
| Tₛ/° C. | 1650 + 1650 | 1650 + 1650 | 1650 + 1650 | 1650 + 1650 | 1650 + 1650 | 1650 + 1650 |
| tₛ/min. | 60 + 60 | 60 + 60 | 60 + 60 | 60 + 60 | 60 + 60 | 60 + 60 |
| T_{Kb}/° C. | 480 | 480 | 480 | 480 | 480 | 480 |
| t_{Kb}/min | 10 | 10 | 10 | 10 | 10 | 10 |
| T_{C1}/° C. | 950 | 950 | 950 | 950 | 950 | 900 |
| t_{C1}/min. | 10 | 10 | 10 | 10 | 10 | 10 |
| T_{C2}/° C. | | | | | | |
| t_{C2} / min. | | | | | | |
| Main crystal | Li₂Si₂O₅ | Li₂Si₂O₅ | Li₂Si₂O₅ | Li₂Si₂O₅ | Li₂Si₂O₅ | Li₂Si₂O₅ |
| phase (wt.-%) | | | | | | |
| Other crystal | Quartz | | Li₂SiO₃ | Li₂SiO₃ | Cristobalite | Li₂SiO₃ |
| phases (wt.-%) | cristobalite | | | Quartz | | Quartz |
| K_{IC} (MPa*m^{0.5}) | | | | | | |
| Chem. stability | | | | | | |
| (μg/cm²) | | | | | | |
| σ_{Biax} (MPa) | | | | | | |

| | Example no. | | | |
|---|---|---|---|---|
| | 47 | 48 | 49 | 50 (Comp.) |
| Composition | Wt.-% | Wt.-% | Wt.-% | Wt.-% |
| SiO₂ | 76.93 | 78.87 | 78.06 | 78.51 |
| Li₂O | 15.93 | 16.33 | 16.16 | 16.25 |
| SnO₂ | 0.57 | 0.57 | 0.57 | |
| K₂O | 1.77 | 1.79 | 1.78 | 1.79 |
| Al₂O₃ | 4.80 | 1.94 | 1.93 | 1.94 |
| P₂O₅ | | 0.50 | 1.50 | 1.51 |
| Σ | 100.00 | 100 | 100 | 100 |
| T_{g}/° C. | 457.8 | 457.8 | 464.7 | 455.8 |
| Tₛ/° C. | 1650 + 1650 | 1650 + 1650 | 1650 + 1650 | 1650 + 1650 |
| tₛ/min. | 60 + 60 | 60 + 60 | 60 + 60 | 60 + 60 |
| T_{Kb}/° C. | 480 | 480 | 480 | 480 |
| t_{Kb}/min | 10 | 10 | 10 | 10 |
| T_{C1}/° C. | 970 | 950 | 950 | 950 |
| t_{C1}/min. | 10 | 10 | 10 | 10 |
| T_{C2}/° C. | | | | |
| t_{C2} / min. | | | | |
| Main crystal | Li₂Si₂O₅ | Li₂Si₂O₅ | Li₂Si₂O₅ | Li₂Si₂O₅ |
| phase (wt.-%) | | (66.2) | (62.0) | (33.3) |
| Other crystal | Li₂SiO₃ | Li₃PO₄ (0.2) | Li₃PO₄ (2.7) | Li₃PO₄ (1.7) |
| phases (wt.-%) | | Cristobalite (0.3) | Cristobalite (2.8) | Cristobalite (2.0) |
| | | Li₂SiO₃ (0.3) | | |
| K_{IC} (MPa*m^{0.5}) | | | | |
| Chem. stability | | | | |
| (μg/cm²) | | | | |
| σ_{Biax} (MPa) | | 270 ± 26 | 226 ± 18 | 52 ± 8 |

* SnO used as starting material

1. A lithium silicate glass ceramic, which comprises 0.02 to 4.5 wt.-% tin, calculated as SnO₂.

2. The glass ceramic according to claim 1, which comprises 65.0 to 89.0 wt.-% SiO₂.

3. The glass ceramic according to claim 1, which comprises 10.0 to 21.0 wt.-% Li₂O.

4. The glass ceramic according to claim 1, which comprises less than 3.0 wt.-% P₂O₅.

5. The glass ceramic according to claim 1, which comprises 0 to 7.0 wt.-% oxide of monovalent elements Me^{I}₂O selected from the group of K₂O, Na₂O, Rb₂O, Cs₂O and mixtures thereof.

6. The glass ceramic according to claim 1, which comprises 0 to 6.0 wt.-% K₂O

7. The glass ceramic according to claim 1, which comprises 0 to 15.0 wt.-% oxide of divalent elements Me^{II}O selected from the group of CaO, MgO, SrO, ZnO and mixtures thereof.

8. The glass ceramic according to claim 1, which comprises 0 to 12.0 wt.-% oxide of trivalent elements Me^{III}₂O₃ selected from the group of Al₂O₃, B₂O₃, Y₂O₃, La₂O₃ and mixtures thereof.

9. The glass ceramic according to claim 1, which comprises 0.1 to 6.0 wt.-% Al₂O₃.

10. The glass ceramic according to claim 1, which comprises lithium disilicate or lithium metasilicate as main crystal phase.

11. The glass ceramic according to claim 1, which comprises 1.0 to 50.0 wt.-% lithium metasilicate crystals.

12. The glass ceramic according to claim 1, which comprises 50.0 to 90.0 wt.-% lithium disilicate crystals.

13. A starting glass, which comprises the components of the glass ceramic according to claim 1.

14. The starting glass according to claim 13, which comprises nuclei for the crystallization of lithium metasilicate and/or lithium disilicate.

15. A glass ceramic or a starting glass, which comprises the components of the glass ceramic according to claim 1, wherein the glass ceramic and the starting glass are in the form of a powder, a granulate, a blank or a dental restoration.

16. A process for producing the glass ceramic according to claim 1, wherein a starting glass comprising 0.02 to 4.5 wt.-% tin, calculated as SnO₂, is subjected to at least one heat treatment in the range of 800 to 1050° C.

17. The process according to claim 16, wherein
(a) the starting glass is subjected to a heat treatment at a temperature of 400 to 600° C., to form starting glass with nuclei, and
(b) the starting glass with nuclei is subjected to a heat treatment at a temperature of 800 to 1050° C., to form the lithium silicate glass ceramic.

18. A process for producing a dental restoration comprising a bridge, inlay, onlay, veneer, abutment, partial crown, crown or facet, in which the glass ceramic according to claim 1 is given the shape of the desired dental restoration by pressing or machining.
